(19) United States
(12) Patent Application Publication
(10) Pub. No.: US 2026/0021115 A1
(43) Pub. Date: Jan. 22, 2026

(54) Synergistic combinations of Spironolactone, Finasteride, Dutasteride, and Minoxidil

(71) Applicant: Michael Anthony Martinez, San Francisco, CA (US)

(72) Inventor: Michael Anthony Martinez, San Francisco, CA (US)

(21) Appl. No.: 18/777,465

(22) Filed: Jul. 18, 2024

(51) Int. Cl. A61K 31/585; A61K 31/506; A61K 31/58

(52) U.S. Cl. CPC A61K 31/585; A61K 31/506; A61K 31/58

(57) ABSTRACT

A method for the treatment or prevention of the symptoms or effects of hair loss of an animal which comprises treating the animal with a therapeutically effective amount of a combination comprising 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof. A pharmaceutical formulation comprising the same. A patient pack comprising at least one active ingredient selected from 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide, 17β-(N-tert-butylcarbamoyl)-4-aza-5α-androst-1-en-3-one, and 2,4-pyrimidinediamine, 6-(1-piperidinyl)-, 3-oxide, and an information insert containing directions on the use of all three active ingredients together in combination.

### CROSS-REFERENCE TO RELATED APPLICATIONS

Not applicable.

### STATEMENTS REGARDING FEDERALLY SPONSORED RESEARCH AND DEVELOPMENT

Not applicable.

### BACKGROUND

The embodiments of the present invention satisfy the needs of having a better treatment for hair loss.

Information relevant to attempts to address these problems can be found in U.S. Pat. Nos. 6,417,191; 6,960,577; 7,5505,93; 8,563,728; which are not admitted to be prior art with respect to the present invention by their mention in this Background Section. It is desirable to have better apparatuses and/or methods than what is disclosed in the identified references.

A 2014 survey by the International Society of Hair Restoration Surgery found that approximately 35 million men and 21 million women in the United States are affected by hair loss. U.S. Food and Drug Administration (FDA) regimens for hair loss treatment currently consist of topical or oral minoxidil, or oral finasteride.

Finasteride was patented by Merck in 1984. In 1992, the FDA approved 5 mg doses for the treatment of benign prostatic hyperplasia (BPH) or prostate gland enlargement. In 1997, the FDA approved 1 mg doses for the treatment of male pattern hair loss. These 1 mg doses were marketed under the name Propecia.

Dutasteride was patented by GlaxoSmithKline in 1993. In 2001, the FDA approved 0.5 g doses for the treatment of BPH. These 0.5 mg doses were marketed under the name Avodart. It was never approved by the FDA for the treatment of hair loss in the US though it is used off-label. It was approved for the treatment of hair loss in 2009 in South Korea and 2015 in Japan.

Both finasteride and dutasteride are 5-alpha reductase inhibitors (5-ARIs). They inhibit the conversion of testosterone into the hormone dihydrotestosterone, or DHT, that is primarily responsible for male pattern hair loss. DHT can bind to androgen receptors and cause hair follicles to stop producing new hair.

Dutasteride is more effective than finasteride at reducing blood levels of DHT. This may be due to the fact that there are two isoenzymes of 5α-reductase. Finasteride only inhibits the type 2 isoenzyme but dutasteride inhibits both the types 1 and 2 isoenzymes. A 2004 study showed that a 5 mg daily dose of dutasteride reduced DHT levels by 98.4%, whereas a 5 mg daily dose of finasteride reduced DHT levels by 70.8%.

A 2006 study showed that men who used dutasteride ended up with a higher increase in hair count as compared to men who used finasteride. This may be due to the previously stated ability of dutasteride to inhibit two isoenzymes. Dutasteride also has a much longer half-life than finasteride. Dutasteride has a half-life of several months whereas finasteride has a half-life of only a few hours.

A person suffering from hair loss might benefit from taking both finasteride and dutasteride. For example, whereas dutasteride users enjoyed a higher increase in hair count, studies have shown that users of finasteride enjoyed both an increase in hair count and a reduction in hair loss. In one study, the addition of a low-dosage dutasteride treatment to a finasteride treatment resulted in a dramatic increase in hair density.

Nevertheless, combining finasteride and dutasteride is generally discouraged. Even though finasteride inhibits only the type 2 isoenzyme while dutasteride inhibits both the types 1 and 2 isoenzymes, finasteride is more widely prescribed for hair loss: finasteride is FDA approved for treating hair loss, whereas dutasteride is not currently FDA approved for treating hair loss and it has greater side effects.

Another pharmaceutical for treating hair loss is minoxidil. Minoxidil is a vasodilator, an anti-inflammatory agent, a Wnt/B-catenin signaling inducer, and an antiandrogen. Minoxidil facilitates the growth of new hair by increasing blood flow. Thus a person suffering from hair loss might benefit from using both minoxidil, and finasteride or dutasteride or both finasteride and dutasteride. Topical minoxidil is FDA approved for treating hair loss. Oral minoxidil is not FDA approved for treating hair loss.

But for the treatment of hair loss, depending on duration, pattern and severity, post-menopausal women may also be prescribed minoxidil, finasteride, dutasteride, and spironolactone, all together. Pairwise combinations are already known for spironolactone and minoxidil; spironolactone and finasteride; and spironolactone and dutasteride. For the treatment of hair loss, depending on duration, pattern and severity, pre-menopausal women are already prescribed with minoxidil 1.25 mg to 5 mg a day orally; and spironolactone 25 mg to 100 mg a day orally. Menstruating and fertile women can only take minoxidil and spironolactone. Menstruating women cannot take finasteride or dutasteride unless sterilized or hysterectomy. The triplet combination is already known for spironolactone and finasteride and dutasteride. However, combinations are not currently known for spironolactone and minoxidil and finasteride and dutasteride. As previously stated, combining finasteride and dutasteride is generally discouraged. However, if a user is comfortable using dutasteride off-label and tolerant to its side effects, there may be substantial results from combining finasteride with dutasteride to decrease serum DHT levels. Minoxidil could contribute further results by increasing blood flow especially to hair follicles.

### SUMMARY

The embodiments of the present invention relate to therapeutic combinations of 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide, 17β-(N-tert-butylcarbamoyl)-4-aza-5α-androst-1-en-3-one, and 2,4-pyrimidinediamine, 6-(1-piperidinyl)-, 3-oxide which have anti-hair loss activity. The present invention is also concerned with pharmaceutical compositions containing said combinations and their use in the treatment of hair loss.

The embodiments of the present invention comprise a method for the treatment or prevention of the symptoms or effects of hair loss of an animal which comprises treating the animal with a therapeutically effective amount of a combination comprising 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof.

The embodiments of the present invention further comprise a method wherein 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof are present in a ratio of [3.33:1: 3.33:8.33] to [400:1: 9.5:20] by weight.

The embodiments of the present invention further comprise a method wherein 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof are present in a ratio of [83.33:1: 3.33:3.33] to [400:1: 9.5:2.5] by weight. This set of ratios is appropriate when using sublingual 2,4-pyrimidinediamine, 6-(1-piperidinyl)-, 3-oxide.

Optionally, said animal is selected from the group consisting of: a male human, and a post-menopausal female human.

Optionally, the combination is administered simultaneously.

Optionally, the combination is administered sequentially. Optionally, the combination is administered as a single combined formulation.

Optionally, each of 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof is present in an amount from 0.15 to 10.0 mg per unit dosage form.

The embodiments of the present invention further comprise a pharmaceutical formulation comprising 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof, in association with one or more pharmaceutically acceptable carriers therefor.

Optionally, the formulation is in unit dosage. Optionally, the formulation is in the form of a tablet or capsule.

The embodiments of the present invention further comprise a patient pack comprising at least one active ingredient selected from 7α-Acetylthio-17α-hydroxy-3-oxopregn-4-ene-21-carboxylic acid γ-lactone or a physiologically functional derivative thereof, N-[2,5-bis (trifluoromethyl) phenyl]-3-oxo-4-aza-5α-androst-1-ene-17β-carboxamide, 17β-(N-tert-butylcarbamoyl)-4-aza-5α-androst-1-en-3-one, and 2,4-pyrimidinediamine, 6-(1-piperidinyl)-, 3-oxide, and an information insert containing directions on the use of all three active ingredients together in combination.

Unless otherwise indicated all weights of active ingredients are calculated in terms of the drug per se. The desired dose is preferably presented as one dosage daily, but two, three, four, five, six or more sub-doses may be administered at appropriate intervals throughout the day. These sub-doses may be administered in unit dosage forms. Alternatively, if the condition of the recipient so requires, the dose may be administered as a continuous infusion.

The components of the combination which may be referred to as active ingredients may be administered for therapy to an animal e.g. a mammal including a human in a conventional manner.

While it is possible for the active ingredients of the combination to be administered as the raw chemical it is preferable to present them as a pharmaceutical formulation. Pharmaceutical formulations according to the present invention comprise a combination according to the invention together with one or more pharmaceutically acceptable carriers or excipients and optionally other therapeutic agents. The carrier(s) must be acceptable in the sense of being compatible with the other ingredients of the formula and not deleterious to the recipient thereof. When the individual components of the combination are administered separately, they are generally each presented as a pharmaceutical formulation. The references hereinafter to formulations refer unless otherwise stated to formulations containing either the combination or a component thereof.

More commonly these days pharmaceutical formulations are prescribed to the patient in “patient packs” containing the whole course of treatment in a single package, usually a blister pack. Patient packs have an advantage over traditional prescriptions, where a pharmacists divides a patients supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patients packs of each formulation, within a package insert diverting the patient to the correct use of the invention is a desirable additional feature of this invention.

Formulations include those suitable for oral, rectal, nasal, topical (including transdermal, buccal and sublingual), or vaginal administration. The formulations may conveniently be presented in unit dosage form and may be prepared by any methods well known in the art of pharmacy. Such methods represent a further feature of the present invention and include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

Formulations of the present invention suitable for oral administration may be presented as discrete units such as capsules, caplets, caehets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Molded tablets may be made by molding a mixture of the powdered compound moistened with an inert liauid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredients in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier. Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of the active combination with the softened or melted carrier(s) followed by chilling and shaping in moulds.

Preferred unit dosage formulations are those containing a daily dose or daily subdose of the active ingredients, as hereinbefore recited, or an appropriate fraction thereof.

It should be understood that in addition to the ingredients particularly mentioned above the formulations of this invention may include other agents conventional in the art having regard to the type of formulation in question, for example, those suitable for oral administration may include such further agents as sweeteners, thickeners and flavoring agents.

The compounds of the combination of the present invention may be obtained in a conventional manner.

### Some Advantages of the Embodiments of the Invention

The embodiments of the present invention treat hair loss through multiple pathways. There may be synergistic effects from treating through multiple pathways. A user may respond more favorably to one of the pathways.

### BRIEF DESCRIPTION OF THE DRAWINGS

There are no drawings.

### DESCRIPTION

### Definitions

The chemical name for spironolactone is 7α-Acetylthio-17α-hydroxy-3-oxopregn-4-ene-21-carboxylic acid γ-lactone.

The chemical name for dutasteride is N-[2,5-bis(trifluoromethyl)phenyl]-3-oxo-4-aza-5α-androst-1-ene-17β-carboxamide.

The chemical name for finasteride is 17β-(N-tert-butylcarbamoyl)-4-aza-5α-androst-1-en-3-one.

The chemical name for minoxidil is 2,4-pyrimidinediamine, 6-(1-piperidinyl)-, 3-oxide.

A post-menopausal woman is a woman whose menstrual period has been gone for longer than 12 consecutive months.

The preferred embodiments and best modes of using the present invention are as follows.

The preferred ranges of prescription dosages for the combination of the four drugs for use by post-menopausal women are: spironolactone 0.5 mg to 200 mg a day oral dose; dutasteride 0.15 mg to 0.5 mg a day oral dose; finasteride 0.5 to 4.75 mg a day oral dose; minoxidil 1.25 mg ranging to 10 mg a day oral dose. The daily oral ratios by weight are [3.33:1:3.33:8.33] to [400:1:9.5:20] for [spironolactone: dutasteride: finasteride: minoxidil].

The preferred ranges of prescription dosages for the combination of the four drugs for use by post-menopausal women are: spironolactone 12.5 mg to 200 mg a day oral dose; dutasteride 0.15 mg to 0.5 mg a day oral dose; finasteride 0.5 to 4.75 mg a day oral dose; sublingual minoxidil 0.5 mg ranging to 1.25 mg a day oral dose. The daily oral ratios by weight are [83.33:1:3.33:3.33] to [400:1 9.5:2.5] for [spironolactone: dutasteride: finasteride: minoxidil].

If a man that wants feminization or male to female transgenderism he may be prescribed all four drugs at above ranges. For spironolactone the main adverse event in men is decreased testosterone, gynecomastia, loss of libido, and erectile dysfunction.

Pre-menopausal woman with sterilization or hysterectomy but still with ovaries may use all 4 drugs with ranges as above for women.

What is claimed is:

1. A method for the treatment or prevention of the symptoms or effects of hair loss of an animal which comprises treating the animal with a therapeutically effective amount of a combination comprising 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof.

2. A method of claim 1, wherein 7α-Acetylthio-17α-hydroxy-3-oxopregn-4-ene-21-carboxylic acid γ-lactone or a physiologically functional derivative thereof, N-[2,5-bis (trifluoromethyl) phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof are present in a ratio of 3.33:1:3.33:8.33 to 400:1:9.5:20 by weight.

3. A method of claim 1, wherein 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof are present in a ratio of 83.33:1:3.33:3.33 to 400:1: 9.5:2.5 by weight.

4. A method according to claim 2, in which the animal is selected from the group consisting of: a male human, and a post-menopausal female human.

5. A method according to claim 2 wherein the combination is administered simultaneously.

6. A method according to claim 2 wherein the combination is administered sequentially.

7. A method according to claim 2 wherein the combination is administered as a single combined formulation.

8. A method according to claim 2 wherein each of 7α-Acetylthio-17α-hydroxy-3-oxopregn-4-ene-21-carboxylic acid γ-lactone or a physiologically functional derivative thereof, N-[2,5-bis (trifluoromethyl) phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof is present in an amount from 0.15 to 10.00 mg per unit dosage form.

9. A pharmaceutical formulation comprising 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide or a physiologically functional derivative thereof, 17β-(N-tert-butylcarbamoyl)-4-aza-5α-androst-1-en-3-one or a physiologically functional derivative thereof, and 2,4-pyrimidinediamine, 6-(1-piperidinyl)-, 3-oxide or a physiologically functional derivative thereof, in association with one or more pharmaceutically acceptable carriers therefor.

10. A formulation according to claim 9 in unit dosage.

11. A formulation according to claim 9 in the form of a tablet or capsule.

12. A patient pack comprising at least one active ingredient selected from 7α-Acetylthio-17α-hydroxy-3-oxopregn-4-ene-21-carboxylic acid γ-lactone or a physiologically functional derivative thereof, N-[2,5-bis(trifluoromethyl)phenyl]-3-oxo-4-aza-5α-androst-1-ene-17β-carboxamide, 17β-(N-tert-butylcarbamoyl)-4-aza-5α-androst-1-en-3-one, and 2,4-pyrimidinediamine, 6-(1-piperidinyl)-, 3-oxide, and an information insert containing directions on the use of all three active ingredients together in combination.
